(19) United States
(12) Patent Application Publication
(10) Pub. No.: US 2012/0197339 A1
(43) Pub. Date: Aug. 2, 2012

(54) TREATMENT DEVICE FOR URINATION DISORDERS

(75) Inventors: Airi Takagi, Kanagawa (JP); Takashi Hisamoto, Kanagawa (JP)

(73) Assignee: PANASONIC CORPORATION, Osaka (JP)

(21) Appl. No.: 13/496,694

(22) PCT Filed: Sep. 10, 2010
(86) PCT No.: PCT/JP2010/005550

§ 371 Date: Mar. 16, 2012

(30) Foreign Application Priority Data
Sep. 17, 2009 (JP) 2009-215361

(51) Int. Cl. A61N 1/36

(52) U.S. Cl. 607/41

(57) ABSTRACT

Disclosed is a treatment device for urination disorders, said treatment device being capable of determining whether or not stimulation-applying electrodes are appropriately arranged and whether or not nerves that control urine accumulation and urination are being appropriately stimulated. The disclosed treatment device can therefore provide appropriate treatment for urination disorders. The treatment device is provided with: at least one pair of stimulation-applying electrodes that apply a stimulation signal to the dorsal surface of the sacrum; a stimulation-signal source that supplies the stimulation signal applied by the stimulation-applying electrodes; a stimulation-transmission detection electrode that is disposed near tissue connected to nerves that run near the sacrum or to the sacrum, and that detects a biosignal that arises in response to the stimulation signal; a ground electrode; a measurement unit that measures the biosignal detected by the stimulation-transmission detection electrode; a control unit that controls the application of the stimulation signal by the pair of stimulation-applying electrodes; and a display means that displays the measurement result from the measurement unit.

### TECHNICAL FIELD

The present invention relates to an urination disorder treatment apparatus for treating an urination disorder.

### BACKGROUND ART

A conventional urination disorder treatment apparatus has a configuration in which a pair of stimulus applying electrodes arranged on the back of the sacral bone with a predetermined distance between them supply a stimulus signal from a stimulus signal supply source. The conventional urination disorder treatment apparatus alleviates an urination disorder by stimulating the urinary bladder and the urethra by the stimulus signal through a nerve which passes through the sacral micturition reflex center and controls collection and discharge of urine.

### CITATION LIST

### Patent Literature

PTL 1

Japanese Patent Application Laid-Open No. 3-26623

### SUMMARY OF THE INVENTION

### Technical Problem

However, the conventional urination disorder treatment apparatus may have a difficulty to perform an appropriate treatment. That is, a pair of stimulus applying electrodes need to be arranged on an appropriate portion on the back of the sacral bone to transmit a stimulus to the nerve which passes through the sacral micturition reflex center and controls collection and discharge of urine. This is because an appropriate stimulus cannot be applied to the urinary bladder and the urethra unless a pair of stimulus applying electrodes are arranged at an appropriate position. However, in the conventional urination disorder treatment apparatus, whether or not the stimulus applying electrodes are arranged at an appropriate position cannot be determined, so that an appropriate treatment may not be performed.

Therefore, an object of the present invention is to provide a urination disorder treatment apparatus which can perform an appropriate treatment of a urination disorder by determining whether the stimulus applying electrodes are appropriately arranged and whether an appropriate stimulus is applied to the nerve that controls collection and discharge of urine.

### Solution to Problem

To achieve the above object, the urination disorder treatment apparatus of the present invention has a configuration described below.
(1) at least a pair of stimulus applying electrodes which is arranged on a back of a sacral bone of the patient with a predetermined distance between the stimulus applying electrodes and which supplies a stimulus signal to the back of the sacral bone;
(2) a stimulus signal supply source that allows the pair of stimulus applying electrodes to supply a stimulus signal;
(3) a stimulus-response detecting electrode which is arranged near a tissue connected to a nerve that passes through the sacral bone or passes near the sacral bone and which detects a biological signal generated in response to the stimulus signal;
(4) a ground electrode arranged on a given portion of skin of the patient;
(5) a measuring section which is connected to the stimulus-response detecting electrode and the ground electrode and which measures a biological signal detected by the stimulus-response detecting electrode;
(6) a control section which is connected to the measuring section and which controls supply of the stimulus signal supplied from the pair of stimulus applying electrodes on the basis of a measurement result of the measuring section; and
(7) a display means which is connected to the control section and on which the measurement result of the measuring section is displayed.

### Advantageous Effects of Invention

According to the urination disorder treatment apparatus of the present invention, it is possible to determine whether or not a pair of stimulus applying electrodes transmit an appropriate stimulus to a nerve which passes through the sacral micturition reflex center and controls collection and discharge of urine. Specifically, whether or not an appropriate stimulus is transmitted can be determined by measuring a biological signal generated in response to a stimulus to the back of the sacral bone using a stimulus-response detecting electrode and a measuring section. Thereby, an effective treatment is realized.

According to the urination disorder treatment apparatus of the present invention, whether or not a pair of stimulus applying electrodes transmit an appropriate stimulus to a nerve which controls collection and discharge of urine can be determined from information displayed on the display means. A therapist (or a patient himself or herself) can apply an appropriate stimulus by changing an arrangement position of a pair of stimulus applying electrodes to an appropriate position on the basis of the information displayed on the display means.

Further, according to the urination disorder treatment apparatus of the present invention, it is possible to adjust the magnitude of the stimulus signal supplied by a pair of stimulus applying electrodes on the basis of the information displayed on the display means. Thereby, a more appropriate stimulus can be applied.

### BRIEF DESCRIPTION OF DRAWINGS

FIG. 1 is a side cross-sectional view of a human body to which a urination disorder treatment apparatus is applied;

FIG. 2 is a rear view of the human body to which the urination disorder treatment apparatus is applied;

FIG. 3 is a block diagram of an urination disorder treatment apparatus in accordance with Embodiment 1 of the present invention;

FIG. 4 is a block diagram of an urination disorder treatment apparatus in accordance with Embodiment 2 of the present invention;

FIG. 5 is a block diagram of a urination disorder treatment apparatus in accordance with Embodiment 3 of the present invention; and

FIG. 6 is a block diagram of an urination disorder treatment apparatus in accordance with Embodiment 4 of the present invention.

### DESCRIPTION OF EMBODIMENTS

First, an outline of a treatment mechanism of an urination disorder by an urination disorder treatment apparatus of the present invention will be described.

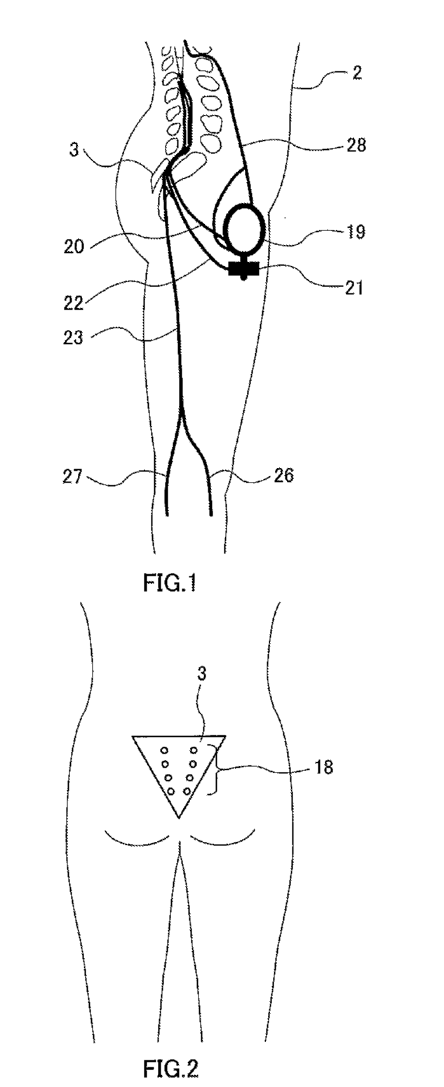

FIG. 1 and FIG. 2 show a region near sacral bone 3 of human body 2. Sacral bone 3 has sacral foramens 18. Sacral foramens 18 have bilaterally symmetrical structure in human body 2. Pelvic splanchnic nerve 20, pudendal nerve 22, and hypogastric nerve 28 are engaged in control of collection and discharge of urine (see FIG. 1). Because these nerves are connected to urine bladder 19 and urethral sphincter muscle 21.

Pelvic splanchnic nerve 20 is connected from sacral foramen 18 to urine bladder 19. Pudendal nerve 22 is connected from sacral foramen 18 to urethral sphincter muscle 21. A symptom of an urination disorder can be alleviated by applying a stimulus to pelvic splanchnic nerve 20 and pudendal nerve 22.

As described later, the urination disorder treatment apparatus of the present invention can apply a stimulus to pelvic splanchnic nerve 20 and pudendal nerve 22 via a pair of stimulus applying electrodes 1a and 1b. The “treatment” by the urination disorder treatment apparatus of the present invention includes alleviating the symptom of the urination disorder.

As shown in FIG. 1 and FIG. 2, sciatic nerve 23 passes through sacral foramen 18 and divides into common peroneal nerve 26 and tibial nerve 27 in a leg. Thereby, sciatic nerve 23 controls a motion of a distal portion of a leg.

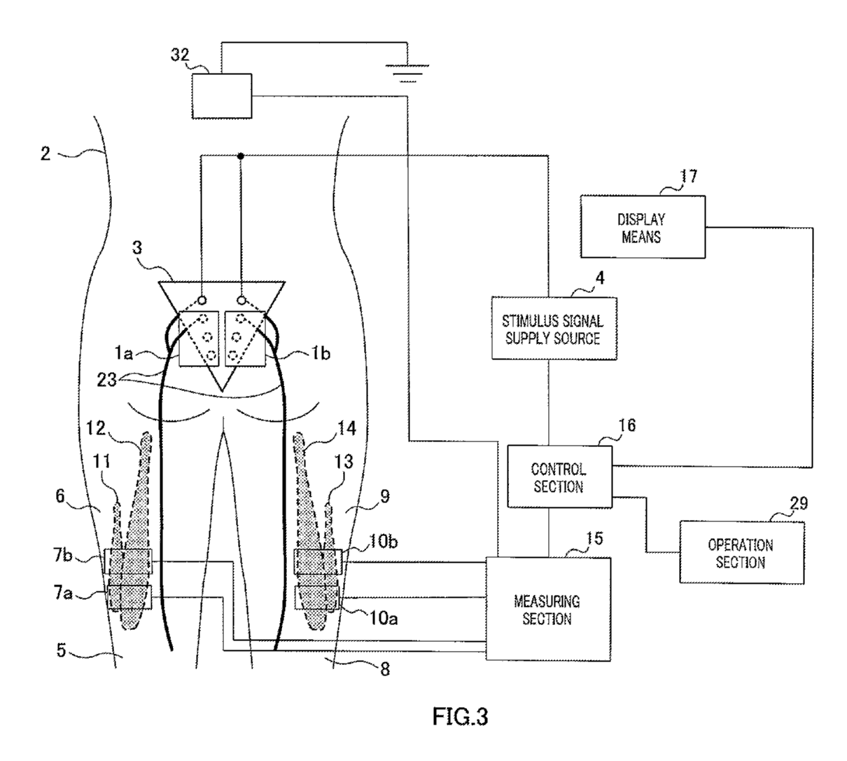

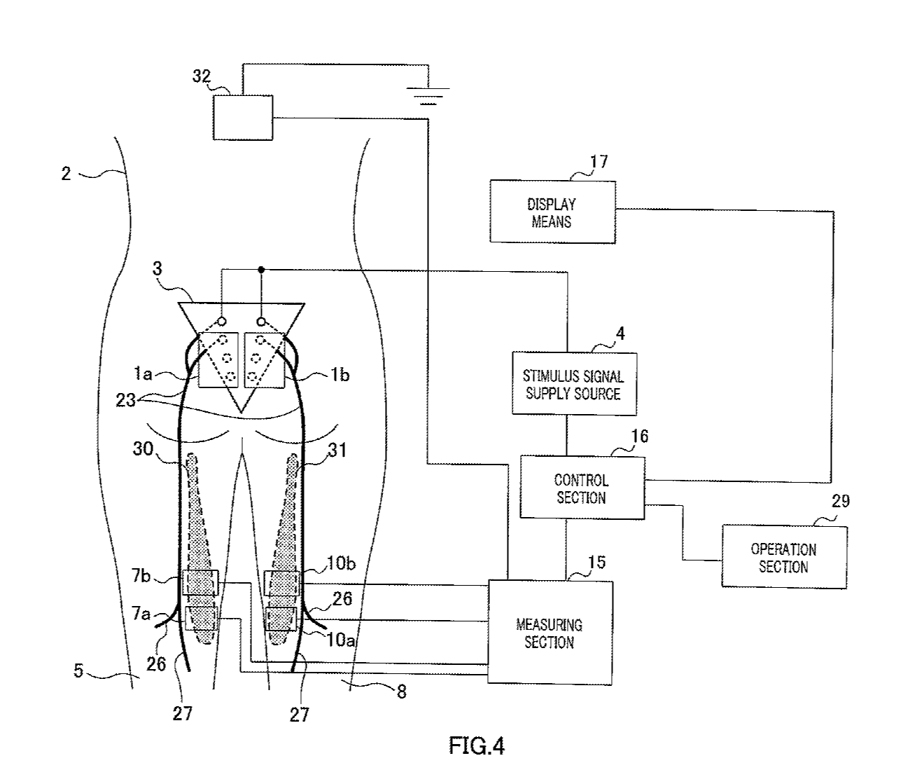

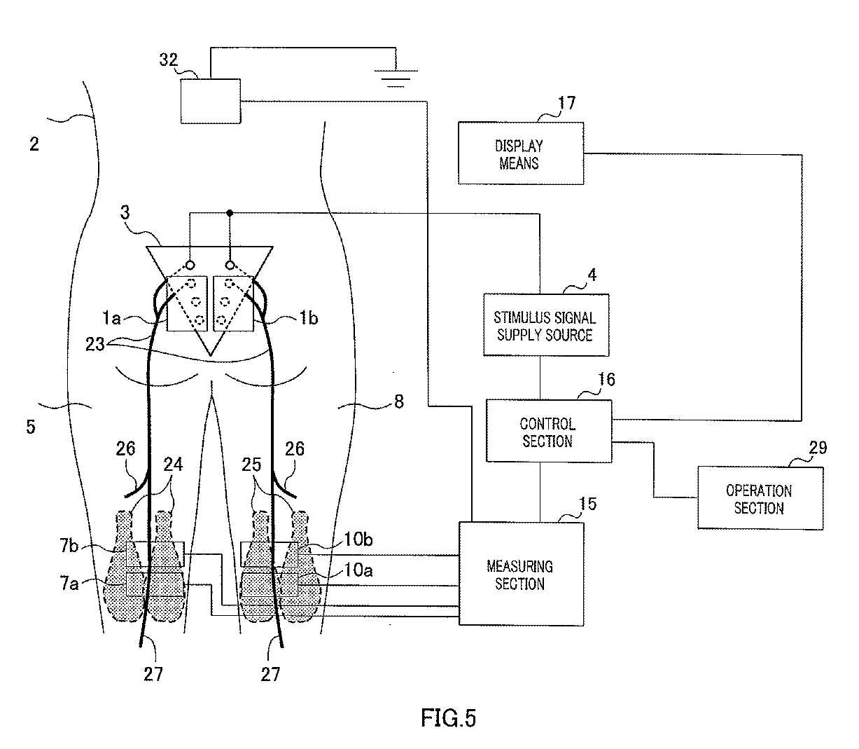

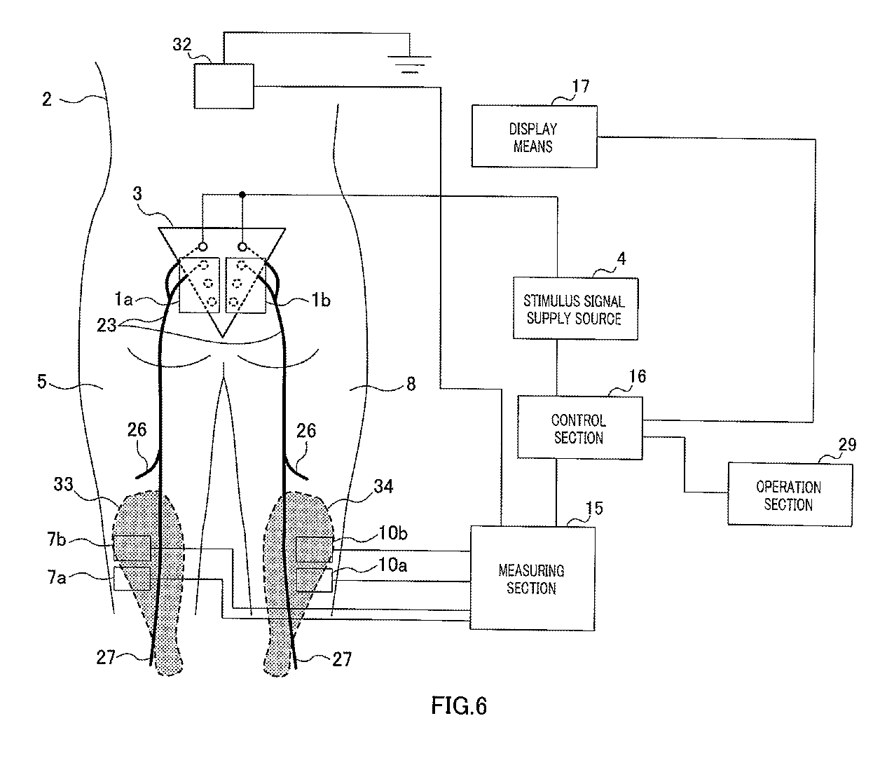

Sciatic nerves 23 have muscle branches and the muscle branches originating from sciatic nerves 23 connect to tissues (muscles) in left leg 5 and right leg 8 of the human body. For example, the muscle branches originating from sciatic nerves 23 connect to biceps femoris muscle 6 of left leg 5 and biceps femoris muscle 9 of right leg 8 (see FIG. 3) and connect to semitendinosus muscle 30 of left leg 5 and semitendinosus muscle 31 of right leg 8 (see FIG. 4). On the other hand, muscle branches originating from tibial nerve 27 connect to gastrocnemius muscle 24 of left leg 5 and gastrocnemius muscle 25 of right leg 8 (see FIG. 5) and connect to soleus muscle 33 of left leg 5 and soleus muscle 34 of right leg 8 (see FIG. 6).

As described later, the urination disorder treatment apparatus of the present invention includes stimulus-response detecting electrodes 7a and 7b and stimulus-response detecting electrodes 10a and 10b (see FIG. 3 and the like) for detecting a biological signal generated in response to a stimulus signal supplied by a pair of stimulus applying electrodes 1a and 1b.

A pair of stimulus-response detecting electrodes 7a and 7b and a pair of stimulus-response detecting electrodes 10a and 10b only have to detect the biological signal generated in response to the stimulus signal supplied by a pair of stimulus applying electrodes 1a and 1b, and the arrangements thereof are not limited. For example, stimulus-response detecting electrodes 7a and 7b and stimulus-response detecting electrodes 10a and 10b may be arranged on skin near urethral sphincter muscle 21 (see FIG. 1) or skin near a perineal muscle that connect to pudendal nerve 22.

However, preferably, stimulus-response detecting electrodes 7a and 7b and stimulus-response detecting electrodes 10a and 10b are arranged on skin (on back of leg) near muscle tissues in a leg, which are tissues to which sciatic nerve 23 connects. The tissues to which sciatic nerve 23 connects also mean tissues that connect to a nerve branching from sciatic nerve 23. The nerve branching from sciatic nerve 23 is, for example, tibial nerve 27.

As shown in FIG. 1 to FIG. 6, sacral foramens 18 have a bilaterally symmetrical structure and sciatic nerves 23 and tibial nerves also have bilaterally symmetrical structures. Therefore, a biological signal generated in response to a stimulus signal to a left sacral foramen can be detected by stimulus-response detecting electrodes 7a and 7b arranged on left leg 5. On the other hand, a biological signal generated in response to a stimulus signal to a right sacral foramen can be detected by stimulus-response detecting electrodes 10a and 10b arranged on right leg 8.

In other words, when a pair of stimulus-response detecting electrodes 7a and 7b and a pair of stimulus-response detecting electrodes 10a and 10b are arranged respectively on skins near muscle tissues in both legs that are tissues connected to sciatic nerves 23, it is easy to separately detect the biological signals generated in response to each of the stimulus signals of a pair of stimulus applying electrodes 1a and 1b. As a result, as described later, it is easy to determine whether or not each of stimulus applying electrodes 1a and 1b is appropriately arranged.

Because of the above reason, it is preferred that stimulus-response detecting electrodes 7a and 7b and stimulus-response detecting electrodes 10a and 10b are arranged near muscles in left leg 5 and right leg 8 respectively.

The biological signal generated in response to each of the stimulus signals of a pair of stimulus applying electrodes 1a and 1b means, typically, an evoked potential. The evoked potential is a potential response that is evoked by tissue when a certain stimulus is applied to a nerve connected to the tissue (for example, muscle tissue).

Hereinafter, examples of embodiments of the present invention will be described. In Embodiments 1 to 4 described below, a form in which stimulus-response detecting electrodes 7a and 7b and stimulus-response detecting electrodes 10a and 10b are arranged near muscle tissues in the legs to which sciatic nerves 23 are connected will be described with reference to the attached drawings.

### Embodiment 1

A urination disorder treatment apparatus according to Embodiment 1 includes a pair of stimulus applying electrodes 1a and 1b, a pair of stimulus-response detecting electrodes 7a and 7b, a pair of stimulus-response detecting electrodes 10a and 10b, ground electrode 32, stimulus signal supply source 4, measuring section 15, control section 16, display means 17, and operation section 29 (see FIG. 3).

As shown in FIG. 3, a pair of stimulus applying electrodes 1a and 1b are arranged on the back of sacral bone 3 symmetrically relative to a central axis (with respect to a central axis) of human body 2 with a predetermined distance in between. The back of the sacral bone means a “surface of the back near the sacral bone”. Stimulus signal supply source 4 is connected to a pair of stimulus applying electrodes 1a and 1b.

As shown in FIG. 3, stimulus-response detecting electrodes 7a and 7b are arranged on the back of biceps femoris muscle 6 of left leg 5, and stimulus-response detecting electrodes 10a and 10b are arranged on the back of biceps femoris muscle 9 of right leg 8. An arrangement distance between stimulus-response detecting electrodes 7a and 7b and an arrangement distance between stimulus-response detecting electrodes 10a and 10b are desired to be set in a range of about 2 to 3 cm.

“Biceps femoris muscle 6” in the present description and the claims includes meanings of short head of biceps femoris muscle 11 and long head of biceps femoris muscle 12. Similarly, “biceps femoris muscle 9” includes meanings of short head of biceps femoris muscle 13 and long head of biceps femoris muscle 14.

Measuring section 15 is connected to stimulus-response detecting electrodes 7a and 7b arranged on biceps femoris muscle 6 and stimulus-response detecting electrodes 10a and 10b arranged on biceps femoris muscle 9.

Ground electrode 32 is arranged on a given skin surface and connected to measuring section 15. Control section 16 is connected to measuring section 15. Stimulus signal supply source 4, display means 17, and operation section 29 are connected to control section 16. Measuring section 15 is connected to stimulus-response detecting electrodes 7a and 7b, stimulus-response detecting electrodes 10a and 10b, and ground electrode 32.

Ground electrode 32 can be arranged on any portion of the skin surface. However, a preferred arrangement position of ground electrode 32 is between a pair of stimulus applying electrodes 1a and 1b and a pair of stimulus-response detecting electrodes 7a and 7b, or between a pair of stimulus applying electrodes 1a and 1b and a pair of stimulus-response detecting electrodes 10a and 10b.

It is preferred that ground electrode 32 is arranged at a position that is not affected by noise caused by muscle action. Although ground electrode 32 shown in FIG. 3 is one electrode having a flat plate shape, two ground electrodes may be symmetrically arranged on left leg 5 and right leg 8 respectively. Ground electrode 32 may have a shape that is wrapped around the trunk of the body or a leg.

A pair of stimulus applying electrodes 1a and 1b can apply a pulse signal of bipolar square wave to the back of sacral bone 3 as a stimulus signal. Stimulus signal supply source 4 is connected to a pair of stimulus applying electrodes 1a and 1b as a supply source of the pulse signal. The stimulus signal (pulse signal) supplied by a pair of stimulus applying electrodes 1a and 1b stimulates a nerve which passes through the micturition reflex center located in sacral bone 3 and controls collection and discharge of urine, so that a urination disorder is alleviated. Although, as described above, examples of the nerve which passes through the micturition reflex center and controls collection and discharge of urine include pelvic splanchnic nerve 20, pudendal nerve 22, and hypogastric nerve 28, preferably, the nerve is pelvic splanchnic nerve 20 and pudendal nerve 22 that pass near the sacral bone.

The voltage of the pulse signal of bipolar square wave, which is a stimulus signal that is supplied from the supply source of stimulus signal supply source 4 and supplied by a pair of stimulus applying electrodes 1a and 1b, can be varied in a range greater than 0 V and smaller than or equal to 80 V. The voltage is set to a maximum value within a range in which patient does not feel pain. The pulse signal of bipolar square wave that is set in this manner has, for example, a pulse width of 200 μs, a frequency of 30 Hz, intermittent timing of 5 seconds of ON and 5 seconds of OFF, and a current of 0 to 80 mA,

The magnitude of the stimulus signal that is supplied from stimulus signal supply source 4 and applied by a pair of stimulus applying electrodes 1a and 1b may be adjusted by control section 16 according to an operation on operation section 29. A therapist (or a patient himself or herself) can operate operation section 29 to set the magnitude of the stimulus signal to an optimal value. Specifically, the therapist can adjust a bipolar square wave voltage of the stimulus signal applied to a pair of stimulus applying electrodes 1a and 1b in a range greater than 0 V and smaller than or equal to 80 V.

In this way, the urination disorder treatment apparatus of Embodiment 1 stimulates the nerve that controls collection and discharge of urine via a pair of stimulus applying electrodes 1a and 1b, so that the function of collecting urine is facilitated, and urinary incontinence and frequent urination are prevented. However, if a pair of stimulus applying electrodes 1a and 1b are not arranged at appropriate positions on the back of sacral bone 3 of human body 2, it is impossible to appropriately stimulate the nerve, so that a sufficient treatment effect cannot be obtained.

Therefore, the urination disorder treatment apparatus of Embodiment 1 includes stimulus-response detecting electrodes 7a and 7b to be arranged on the back of biceps femoris muscle 6 of left leg 5, and stimulus-response detecting electrodes 10a and 10b to be arranged on the back of biceps femoris muscle 9 of right leg 8.

The urination disorder treatment apparatus of Embodiment 1 can measure a potential difference between stimulus-response detecting electrodes 7a and 7b and a potential difference between stimulus-response detecting electrodes 10a and 10b. Specifically, measuring section 15 measures the potential difference between stimulus-response detecting electrodes 7a and 7b and the potential difference between stimulus-response detecting electrodes 10a and 10b on the basis of the potential of ground electrode 32. In other words, measuring section 15 can measure a muscle action potential of biceps femoris muscle 6 of left leg 5 detected by stimulus-response detecting electrodes 7a and 7b and a muscle action potential of biceps femoris muscle 9 of right leg 8 detected, by stimulus-response detecting electrodes 10a and 10b.

On the other hand, when a pair of stimulus applying electrodes 1a and 1b stimulate nerves, muscle actions of biceps femoris muscles 6 and 9 are evoked. The feature of the urination disorder treatment apparatus of Embodiment 1 is that the evoked muscle action potentials (referred to as “evoked potentials”) of biceps femoris muscles 6 and 9 are detected by stimulus-response detecting electrodes 7a and 7b and stimulus-response detecting electrodes 10a and 10b as a biological signal and the biological signal is measured by measuring section 15.

The evoked potentials, which are biological signals, are detected by stimulus-response detecting electrodes after 10 ms to 30 ms has elapsed from when a pair of stimulus applying electrodes 1a and 1b applied a stimulus signal to the back to sacral bone 3.

In this way, it is possible to determine whether or not a pair of stimulus applying electrodes 1a and 1b apply an appropriate stimulus on the basis of the potentials (evoked potentials) detected by stimulus-response detecting electrodes 7a and 7b and stimulus-response detecting electrodes 10a and 10b. In other words, it is possible to determine whether or not a pair of stimulus applying electrodes 1a and 1b are arranged at appropriate positions on the back of sacral bone 3.

If one electrode 1a of a pair of stimulus applying electrodes is not arranged at an appropriate position on the back of sacral bone 3, even when a pair of stimulus applying electrodes 1a and 1b apply a stimulus signal, the stimulus is not appropriately transmitted to biceps femoris muscle 6 of left leg 5 via sciatic nerve 23. Therefore, the biological signal is not detected by stimulus-response detecting electrodes 7a and 7b, or only a small biological signal is detected by stimulus-response detecting electrodes 7a and 7b.

Similarly, if the other electrode 1b of the pair of stimulus applying electrodes is not arranged at an appropriate position on the back of sacral bone 3, even when a pair of stimulus applying electrodes 1a and 1b apply an electrical stimulus, the stimulus is not appropriately transmitted to biceps femoris muscle 9 of right leg 8 via sciatic nerve 23. Therefore, the biological signal is not detected by stimulus-response detecting electrodes 10a and 10b, or only a small biological signal is detected by stimulus-response detecting electrodes 10a and 10b.

The biological signal measured by measuring section 15 is transmitted to control section 16. Control section 16 displays a measurement result measured by measuring section 15 on display means 17. The measurement result displayed on display means 17 may be a measurement value itself of the biological signal measured by measuring section 15, a determination whether or not an appropriate stimulus is transmitted to a nerve passing through the sacral bone or passing near the sacral bone based on the measurement value, or a determination whether or not a pair of stimulus applying electrodes 1a and 1b are arranged at appropriate positions on the back of sacral bone 3.

The determination whether or not an appropriate stimulus is transmitted, or the determination whether or not the stimulus applying electrodes are appropriately arranged is performed by the methods described below.

(1) A lower limit value of the magnitude (potential difference) of the biological signal to be measured is set in advance, and control section 16 performs the determination on the basis of whether or not the magnitude (potential difference) of the actually measured biological signal exceeds the lower limit value.

(2) A time period (latent time) until the biological signal is measured is set in advance, and control section 16 performs the determination on the basis of whether or not the actually measured biological signal is measured in the set time period (latent time). The time period until the biological signal is measured is desired to be set to a time period from a time (0 ms) when a stimulus is applied to the back of sacral bone 3 by the stimulus applying electrodes to a time when about 10 to 30 ms will have elapsed.

For example, if a biological signal detected by stimulus-response detecting electrodes 7a and 7b arranged on left leg 5 is measured extremely earlier than or later than the set time period (latent time), control section 16 displays that the stimulus transmission from stimulus applying electrode 1a is inappropriate on display means 17 or displays that the arrangement of stimulus applying electrode 1a is inappropriate on display means 17.

On the other hand, if the biological signal detected by stimulus-response detecting electrodes 7a and 7b is measured around the set time period (latent time), the magnitude (potential difference) of the measured biological signal is compared with the set lower limit value of the magnitude (potential difference) of the biological signal.

When the magnitude of the measured biological signal is smaller than or equal to the set lower limit value, control section 16 displays that the stimulus transmission from stimulus applying electrode 1a is inappropriate on display means 17 or displays that the arrangement of stimulus applying electrode 1a is inappropriate on display means 17. On the other hand, when the magnitude of the measured biological signal is greater than the lower limit value set in control section 16, control section 16 displays that the stimulus transmission from stimulus applying electrode 1a is appropriate on display means 17 or displays that the arrangement of stimulus applying electrode 1a is appropriate on display means 17.

Similarly, if a biological signal detected by stimulus-response detecting electrodes 10a and 10b arranged on right leg 8 is measured extremely earlier than or later than the set time period (latent time), control section 16 displays that the stimulus transmission from stimulus applying electrode 1b is inappropriate on display means 17 or displays that the arrangement of stimulus applying electrode 1b is inappropriate on display means 17.

If the biological signal detected by stimulus-response detecting electrodes 10a and 10b is measured around the set time period (latent time), the magnitude (potential difference) of the measured biological signal is compared with the set lower limit value of the magnitude (potential difference) of the biological signal.

When the magnitude of the measured biological signal is smaller than or equal to the set lower limit value, control section 16 displays that the stimulus transmission from stimulus applying electrode 1b is inappropriate on display means 17 or displays that the arrangement of stimulus applying electrode 1b is inappropriate on display means 17. On the other hand, when the magnitude of the measured biological signal is greater than the lower limit value set in control section 16, control section 16 displays that the stimulus transmission from stimulus applying electrode 1b is appropriate on display means 17 or displays that the arrangement of stimulus applying electrode 1b is appropriate on display means 17.

It may be difficult to determine whether the signal detected by measuring section 15 is a biological signal generated in response to the stimulus signal or noise. Therefore, measuring section 15 may measure the signal a plurality of times. Specifically, there may be a process in which the biological signal detected by stimulus-response detecting electrodes 7a and 7b and stimulus-response detecting electrodes 10a and 10b is measured a plurality of times and a plurality of measured values are averaged. In this case, whether or not an appropriate stimulus is applied or whether or not the stimulus applying electrode is appropriately arranged is determined on the basis of whether or not the averaged biological signal is measured in the set time period (latent time) and whether or not the magnitude of the averaged biological signal is greater than the set lower limit value of the magnitude (potential difference) of the biological signal.

Further, in a treatment by the urination disorder treatment apparatus of Embodiment 1, a biological signal X detected by stimulus-response detecting electrodes 7a and 7b arranged on left leg 5 may be compared with a biological signal Y detected by stimulus-response detecting electrodes 10a and 10b arranged on right leg 8. Specifically, a time period (latent time) until when the biological signal X is detected may be compared with a time period (latent time) until when the biological signal Y is detected, or the magnitude (potential difference) of the biological signal X may be compared with the magnitude (potential difference) of the biological signal Y. If there is a large difference between the compared latent times or between the compared potential differences, the arrangement state of a pair of stimulus applying electrodes 1a and 1b is adjusted. Thereby, a pair of stimulus applying electrodes 1a and 1b are arranged more appropriately, so that it is possible to improve treatment effect.

As described above, control section 16 can adjust the magnitude of the stimulus signal applied by a pair of stimulus applying electrodes 1a and 1b according to an operation on operation section 29. The magnitude of the biological signal detected by stimulus-response detecting electrodes 7a and 7b and the magnitude of the biological signal detected by stimulus-response detecting electrodes 10a and 10b can be greater than the set lower limit value by adjusting the magnitude of the stimulus signal.

The magnitude of the stimulus signal is adjusted according to an individual patient. Because an appropriate magnitude of the stimulus signal to obtain treatment effect on alleviating urination disorder is different for each patient. Therefore, it is preferred to adjust the magnitude of the bipolar square wave pulse signal applied by a pair of stimulus applying electrodes 1a and 1b by checking treatment effect of each patient.

A user can check whether or not an appropriate stimulus is transmitted on the basis of information displayed on display means 17. In other words, the user can perform treatment while the user realizes the treatment effect by the stimulus applied through a pair of stimulus applying electrodes 1a and 1b.

Hereinafter, embodiments in which the stimulus-response detecting electrodes are arranged differently from those in Embodiment 1 will be described in Embodiments 2 to 4. On the other hand, components other than the stimulus-response detecting electrodes of the urination disorder treatment apparatus of Embodiments 2 to 4 are the same as those in the urination disorder treatment apparatus in Embodiment 1, so that the description thereof will be omitted.

### Embodiment 2

FIG. 4 shows an urination disorder treatment apparatus in accordance with Embodiment 2 of the present invention. The stimulus-response detecting electrodes of the urination disorder treatment apparatus of Embodiments 2 are arranged on the backs of semitendinosus muscles 30 and 31 located inside of biceps femoris muscles 6 and 9 (see FIG. 3). Specifically, stimulus-response detecting electrodes 7a and 7b are arranged on the back of semitendinosus muscle 30 of left leg 5, and stimulus-response detecting electrodes 10a and 10b are arranged on the back of semitendinosus muscle 31 of right leg 8.

As shown in FIG. 1 and FIG. 2, sciatic nerves 23 pass through sacral foramens 18. As shown in FIG. 4, muscle branches originating from sciatic nerves 23 connect to semitendinosus muscle 30 of left leg 5 and semitendinosus muscle 31 of right leg 8.

Therefore, it is possible to determine whether the arrangement state of a pair of stimulus applying electrodes 1a and 1b is appropriate or not by measuring biological signals detected by stimulus-response detecting electrodes 7a and 7b arranged on the back of semitendinosus muscle 30 of left leg 5 and stimulus-response detecting electrodes 10a and 10b arranged on the back of semitendinosus muscle 31 of right leg 8. As described above, measuring the biological signal includes measuring the latent time and the magnitude (potential difference) of the biological signal.

### Embodiment 3

FIG. 5 shows an urination disorder treatment apparatus in accordance with Embodiment 3 of the present invention. The stimulus-response detecting electrodes of the urination disorder treatment apparatus of Embodiments 3 are arranged on the backs of gastrocnemius muscles 24 and 25 located in more distal portions of the legs than biceps femoris muscles 6 and 9 (see FIG. 3) and semitendinosus muscles 30 and 31 (see FIG. 4). Specifically, stimulus-response detecting electrodes 7a and 7b are arranged on the back of gastrocnemius muscle 24 of left leg 5, and stimulus-response detecting electrodes 10a and 10b are arranged on the back of gastrocnemius muscle 25 of right leg 8.

As shown in FIG. 1 and FIG. 2, sciatic nerves 23 pass through sacral foramens 18. As shown in FIG. 5, sciatic nerve 23 divides into common peroneal nerve 26 and tibial nerve 27 in a lower portion. The muscle branches of tibial nerves 27 arc connected to gastrocnemius muscles 24 and 25 respectively.

Therefore, it is possible to determine whether the arrangement state of a pair of stimulus applying electrodes 1a and 1b is appropriate or not by measuring biological signals detected by stimulus-response detecting electrodes 7a and 7b arranged on the back of gastrocnemius muscle 24 of left leg 5 and stimulus-response detecting electrodes 10a and 10b arranged on the back of gastrocnemius muscle 25 of right leg 8. As described above, measuring the biological signal includes measuring the latent time and the magnitude (potential difference) of the biological signal.

### Embodiment 4

FIG. 6 shows an urination disorder treatment apparatus in accordance with Embodiment 4 of the present invention. The stimulus-response detecting electrodes of the urination disorder treatment apparatus of Embodiments 4 are arranged on the backs of posterolateral soleus muscles 33 and 34 located in more distal portions than gastrocnemius muscles 24 and 25 (see FIG. 5). Specifically, stimulus-response detecting electrodes 7a and 7b are arranged on the back of soleus muscle 33 of left leg 5, and stimulus-response detecting electrodes 10a and 10b are arranged on the back of soleus muscle 34 of right leg 8.

As shown in FIG. 1 and FIG. 2, sciatic nerves 23 pass through sacral foramens 18. As shown in FIG. 6, sciatic nerve 23 divides into common peroneal nerve 26 and tibial nerve 27 in a lower portion. The muscle branches of tibial nerves 27 are connected to soleus muscles 33 and 34 respectively.

Therefore, it is possible to determine whether the arrangement state of a pair of stimulus applying electrodes 1a and 1b is appropriate or not by measuring biological signals detected by stimulus-response detecting electrodes 7a and 7b arranged on the back of soleus muscle 33 of left leg 5 and stimulus-response detecting electrodes 10a and 10b arranged on the back of soleus muscle 34 of right leg 8. As described above, measuring the biological signal includes measuring the latent time and the magnitude (potential difference) of the biological signal.

### INDUSTRIAL APPLICABILITY

As described above, the urination disorder treatment apparatus of the present invention treats a urination disorder by transmitting a stimulus signal to a nerve which passes through the sacral micturition reflex center and controls collection and discharge of urine through at least a pair of stimulus applying electrodes arranged on the back of the sacral bone with a predetermined distance between them. The urination disorder treatment apparatus of the present invention includes the stimulus-response detecting electrodes and can detect a biological signal generated in response to the stimulus signal. It is possible to display whether or not an appropriate biological signal is given on the display means by measuring the biological signal. The therapist can appropriately adjust the arrangement position of a pair of stimulus applying electrodes so that an appropriate stimulus signal is applied while checking the display. Thereby, an appropriate treatment is realized.

When the urination disorder treatment apparatus of the present invention includes an operation section for adjusting the strength of the stimulus signal, the therapist can adjust the strength of the stimulus signal by operating the operation section to apply an appropriate stimulus signal while checking the display, so that the therapist can apply a stimulus appropriate for the patient.

As described above, the urination disorder treatment apparatus of the present invention can realize a more effective treatment of urination disorder.

This application claims the priority of Japanese Patent Application No. 2009-215361 filed Sep. 17, 2009, which is hereby incorporated by reference herein in its entirety, including the specification, claims, and drawings.

### Reference Signs List

1a, 1b A pair of stimulus applying electrodes
2 Human body
3 Sacral bone
4 Stimulus signal supply source
5 Left leg
6, 9 Biceps femoris muscle
7a, 7b stimulus-response detecting electrode
8 Right leg
10a, 10b stimulus-response detecting electrode
11, 13 Short head of biceps femoris muscle
12, 14 Long head of biceps femoris muscle
15 Measuring section
16 Control section
17 Display means
18 Sacral foramens
19 Urine bladder
20 Pelvic splanchnic nerve
21 Urethral sphincter muscle
22 Pudendal nerve
23 Sciatic nerve
24, 25 Gastrocnemius muscle
26 Common peroneal nerve
27 Tibial nerve
28 Hypogastric nerve
29 Operation section
30, 31 Semitendinosus muscle
32 Ground electrode
33, 34 Soleus muscle

1-10. (canceled)

11. A urination disorder treatment apparatus for treating a urination disorder of a patient, the urination disorder treatment apparatus comprising:
at least a pair of stimulus applying electrodes which is arranged on a back of a sacral bone of the patient with a predetermined distance between the stimulus applying electrodes and which supplies a stimulus signal to the back of the sacral bone;
a stimulus signal supply source that causes the pair of stimulus applying electrodes to supply a stimulus signal;
a stimulus-response detecting electrode which is arranged near a tissue connected to a nerve that passes through the sacral bone or passes near the sacral bone and which detects a biological signal generated in response to the stimulus signal;
a ground electrode arranged on a given portion of skin of the patient;
a measuring section which is connected to the stimulus-response detecting electrode and the ground electrode and which measures a biological signal detected by the stimulus-response detecting electrode;
a control section which is connected to the measuring section and which controls supply of the stimulus signal supplied from the pair of stimulus applying electrodes on the basis of a measurement result of the measuring section; and
a display section which is connected to the control section and on which the measurement result of the measuring section is displayed.

12. The urination disorder treatment apparatus according to claim 11, wherein the measuring section measures an evoked potential that is evoked corresponding the stimulus signal supplied to the back of the sacral bone from the stimulus applying electrodes.

13. The urination disorder treatment apparatus according to claim 11, wherein the nerve that passes through the sacral bone or passes near the sacral bone is any one of a sciatic nerve, a tibial nerve, and a pudendal nerve.

14. The urination disorder treatment apparatus according to claim 13, wherein two stimulus-response detecting electrodes are arranged on a back of a biceps femoris muscle connected to the sciatic nerve in left leg and two stimulus-response detecting electrodes are arranged on a back of a biceps femoris muscle connected to the sciatic nerve in right leg.

15. The urination disorder treatment apparatus according to claim 13, wherein two stimulus-response detecting electrodes are arranged on a back of a semitendinosus muscle connected to the sciatic nerve in left leg and two stimulus-response detecting electrodes are arranged on a back of a semitendinosus muscle connected to the sciatic nerve in right leg.

16. The urination disorder treatment apparatus according to claim 13, wherein two stimulus-response detecting electrodes are arranged on a back of a gastrocnemius muscle connected to the tibial nerve in left leg and two stimulus-response detecting electrodes are arranged on a back of a gastrocnemius muscle connected to the tibial nerve in right leg.

17. The urination disorder treatment apparatus according to claim 13, wherein two stimulus-response detecting electrodes are arranged on a back of a soleus muscle connected to the tibial nerve in left leg and two stimulus-response detecting electrodes are arranged on a back of a soleus muscle connected to the tibial nerve in right leg.

18. The urination disorder treatment apparatus according to claim 13, wherein the stimulus-response detecting electrode is arranged on skin near a urethral sphincter muscle or skin near a perineal muscle that are connected to the pudendal nerve.

19. The urination disorder treatment apparatus according to claim 11, wherein the control section controls a magnitude of the stimulus signal supplied by the stimulus applying electrodes on the basis of a magnitude of the biological signal measured by the measuring section.

20. The urination disorder treatment apparatus according to claim 11, wherein the control section performs display on the display section on the basis of a magnitude of the biological signal measured by the measuring section.
